(19) United States
(12) Patent Application Publication
(10) Pub. No.: US 2006/0229661 A1
(43) Pub. Date: Oct. 12, 2006

(54) Device for building up muscle

(76) Inventor: Yoshiaki Sato, Tokyo (JP)

Correspondence Address: PILLSBURY WINTHROP SHAW PITTMAN LLP, 1650 TYSONS BOULEVARD, MCLEAN, VA, 22102, US

(21) Appl. No.: 10/552,991

(22) PCT Filed: Apr. 13, 2004
(86) PCT No.: PCT/JP04/05249

§ 371 Date: Oct. 13, 2005

(30) Foreign Application Priority Data
Apr. 15, 2003 (JP) 2003-110903

(51) Int. Cl. A61B 17/00

(52) U.S. Cl. 606/201

(57) ABSTRACT

To provide a muscle development device wherein a sufficient pressure can be applied to every part of the limb intended to be compressed, even under the influence of muscle movements.

To provide a muscle development device comprising a hollow tight fitting band 1 having a tube 5 and a wire-like piece 6 provided therein; and fastening means for use in keeping a length of the tight fitting band 1 in a loop having a desired size, the muscle development device being used to develop muscles of a limb while restricting the blood flow therethrough by means of applying, with the tight fitting band 1 being wrapped around a predetermined compressed range of the limb and the tight fitting band 1 being fastened with the fastening means to have a desired size, a predetermined pressure to the limb around which the tight fitting band 1 is wrapped, the pressure being produced by introducing air to the tube 5. The wire-like piece 6 is designed to limit the direction towards which the tube 5 is allowed to inflate as the tube 5 is filled with air, to against the muscles as determined with the tight fitting band 1 being rest on the muscles.

### TECHNICAL FIELD

The present invention relates to a muscle development device for the muscle development. More particularly, the present invention relates to a muscle development device suitable for a pressure muscle development method that allows healthy people as well as people having motor abnormalities to develop their muscles in an effective manner.

### BACKGROUND OF THE INVENTION

The present inventor has conducted studies for some time in order to work out a muscle development method for easy, safe, and effective muscle development, and put together the accomplishments into a patent application having Japanese Patent Application No. 5-313949, which has been granted as Japanese Patent No. 2670421.

The muscle development method according to the subject patent, which involves the application of pressure, is a distinctive non-conventional one called a “Pressure Muscle Development Method”. This muscle development method is based on the following theoretical concept.

Muscles are composed of slow-twitch muscle fibers and fast-twitch muscle fibers. Slow-twitch muscle fibers are limited in their potential for growth. Accordingly, it is necessary to recruit fast-twitch muscle fibers of the slow- and fast-twitch muscle fibers in order to develop muscles. Recruitment of fast-twitch muscle fibers causes lactic acid buildup in the muscles, which triggers secretion of growth hormone from the pituitary. The growth hormone has effects of, for example, promoting muscle growth and shedding body fat. This means that recruitment of fast-twitch muscle fibers results in development of fast-twitch muscle fibers and, in turn, the entire muscles.

Slow-twitch muscle fibers and fast-twitch muscle fibers are different from each other in terms of the following. Slow-twitch muscle fibers use oxygen for energy and are recruited for low-intensity activities. Fast-twitch muscle fibers provide for activities regardless of whether or not oxygen is present. They are recruited after the slow-twitch muscle fibers for highly intense activities. Therefore, it is necessary to cause the earlier recruited slow-twitch muscle fibers to be exhausted soon in order to recruit fast-twitch muscle fibers.

Conventional muscle development methods use exercises with, for example, a barbell to cause the slow-twitch muscle fibers to be exhausted first, and then to recruit the fast-twitch muscle fibers. This requires a significant amount of exercises, is time-consuming, and tends to increase the burden on muscles and joints.

On the other hand, when a certain range of muscles is compressed to restrict the blood flow through the muscles that is more distal than the compressed range before muscle exercises, less oxygen is supplied to these muscles. The slow-twitch muscle fibers, which require oxygen for energy, are thus exhausted in a short period of time. Muscle exercises with blood-flow restriction by application of pressure will result in recruitment of the fast-twitch muscle fibers without needing a large amount of exercises.

In addition, restriction of the blood flow by application of pressure makes the lactic acid built up in the muscles less likely to be removed from the muscles. Thus, the muscle lactic acid level is more likely to rise and a much larger amount of growth hormone is secreted, as compared with the case where the blood flow is unrestricted.

Based on this theoretical concept, to restrict the muscle blood flow can provide significant development of the muscles.

The muscle development method according to the aforementioned patent is premised on the theoretical concept of the muscle development by the restriction of blood flow. More specifically, a compression pressure for the blood flow restriction is applied to a range near the muscles that you want to develop and closer to the heart, i.e., a proximal region near those muscles. The compression pressure is controlled to put an appropriate stress attributed to the reduced blood flow on the muscles, thereby causing muscle fatigue. In this way, effective muscle development is achieved.

This muscle development method has a significant feature in muscle development not necessarily requiring exercises because it involves developing muscles by putting a stress attributed to a reduced blood flow on the muscles. In addition, this muscle development method can compensate for a total amount of stress that is placed on the muscles by putting a stress attributed to a reduced blood flow on the muscles. When combined with some exercises, the method advantageously provides a less exercise-related stress than conventionally. This advantage brings about some effects: the possibility of incurring damages to the joints or muscles can be reduced and the period of training can be shortened, as a result of decrease in amount of muscle exercises.

The implementation of this muscle development method requires a muscle development device that can restrict the blood flow through the muscles that you want to develop and can provide accurate control of a compression force being applied to the muscles.

An example of devices that can be used for the restriction of blood flow through muscles includes a tight fitting device which restricts the blood flow with a compression force produced with a belt, as disclosed by the present inventor in the aforementioned Japanese Patent Application No. 5-313949.

This tight fitting device has an advantage of being able to be narrowed, which makes it possible to fit the device exactly around an appropriate range on the muscles through which the blood flow is intended to be restricted. However, it is difficult to determine the exact measure of a compression pressure applied to the muscles. Having the exact measure of the compression pressure available would cause a nuisance of somewhat high costs of manufacture.

The present inventor has made studies for a muscle development device that overcomes the aforementioned problems.

In the course of these studies, the present inventor made an invention relating to a muscle development device as disclosed in Japanese Patent Application No. 8-248317. This invention comprises a tight fitting band having a rubber-made tube provided therein. It lies in an effective use of a modified version of a blood pressure cuff in which a user puts the tight fitting band around his or her arm and a compression force is applied to the arm by introducing air into the tube.

Blood pressure cuffs have an advantage in that the compression force can be controlled easily by changing the air pressure. However, the tight fitting band used by being rest on the muscles is wider than necessary. This means the problem of impossibility of fitting it exactly above the desired region of muscles that you want to develop. In addition, a relatively wide tight fitting band covers a larger area on the muscles when the blood pressure cuff is fitted above the region of muscles. The tight fitting band hinders the contraction of muscles during exercises. Therefore, exercises with such a tight fitting band are rather difficult.

Taking the above into consideration, it is desirable to narrow the tight fitting band of the blood pressure cuff, but this also reduces the width of the tube provided in the space within the tight fitting band accordingly. When the air is introduced into it for the application of pressure, the tube inflates in a direction away from the muscles (outwards). The pressure produced by the inflation of the tube would disadvantageously be directed outwards rather than being applied to the muscles well, resulting in a failure of application of an appropriate compression force to the muscles.

The muscle development device disclosed in the above-mentioned Japanese Patent Application No. 8-248317, which was devised in order to overcome such drawbacks, comprises an flat piece that limits the direction towards which the tube is allowed to inflate, to against the muscles (inwards).

The muscle development device according to the subject invention uses the aforementioned flat piece that makes it possible to limit the direction towards which the tube is allowed to inflate, to inward. The exact measure of a compression pressure can be determined during the use of the muscle development device. This significantly contributes to the widespread use of the muscle development method by the restriction of the blood flow.

However, such a muscle development device also has a challenge to be solved.

This is due to the muscle development device in question uses a plate-shaped flat piece.

More specifically, the flat piece is a plate-like object having a certain length. It “bridges” between the ups of undulating muscles as shown in FIG. 11. There is a gap between the muscles and a tight fitting band 101, and a portion thereof does not contact with the surface of the muscles. This would sometimes give rise to uneven compression by the pressure and possible unavailability of uniform pressure to the limb intended to be compressed.

An object of the present invention is to provide a muscle development device wherein the direction towards which the tube is allowed to inflate is limited to inward and a sufficient pressure can be applied to every part of the limb intended to be compressed, even under the influence of muscle movements.

### SUMMARY OF THE INVENTION

In order to achieve the aforementioned objects, the present invention provides a muscle development device comprising a hollow tight fitting band (having, for example, a shape of a long hollow object or a long thin bag) having a tube and a wire-like piece provided therein; and fastening means for use in keeping a length of the tight fitting band in a loop having a desired size, the muscle development device being used to develop muscles of a limb while restricting the blood flow therethrough by means of applying, with said tight fitting band being wrapped around a predetermined compressed range of said limb and said tight fitting band being fastened with said fastening means to have a desired size, a predetermined pressure to said limb around which said tight fitting band is wrapped, the pressure being produced by introducing air to said tube, said wire-like piece being designed to limit the direction towards which said tube is allowed to inflate as said tube is filled with air, to against the muscles as determined with said tight fitting band being rest on the muscles.

The muscle development device according to the present invention uses a wire-like piece rather than an flat piece as a means to limit the direction towards which said tube is allowed to inflate as the tube is filled with air, to against the muscles as determined with said tight fitting band being rest on the muscles (it may be referred to as “inward” in this specification).

A thin wire-like piece is used that can more easily hold the surface of complex moving muscles in closer contact with it than the plate-like object can do. Therefore, a sufficient pressure can be applied to every part of the limb intended to be compressed, even under the influence of muscle movements.

Said wire-like piece may have any one of suitable configurations as long as it is designed to limit the direction towards which said tube is allowed to inflate as said tube is filled with air, to against the muscles as determined with said tight fitting band being rest on the muscles.

For example, the wire-like piece may have a segment that is not parallel to the lengthwise direction of said tight fitting band.

With the wire-like piece so configured, the segment that is not parallel to the lengthwise direction of said tight fitting band holds the tube properly as the tight fitting band is rest on the muscles. The tight fitting band can thus hold a complex up-and-down surface of the muscles in close contact with it.

The wire-like piece having the segment that is not parallel to the lengthwise direction of the tight fitting band is a set of wire-like pieces that are placed in a direction not parallel to the lengthwise direction of said tight fitting band at a predetermined distance along the length of said tight fitting band. Alternatively, the wire-like piece may be a single wire-like piece, the single wire-like piece may be bent into a zigzag pattern like teeth of a saw or into meander with a series of regular bends like a shape obtained by straightening a circumferential contour of gear teeth.

For the set of wire-like pieces, adjacent wire-like pieces are away from each other at a certain distance. The portions of the tight fitting band that fall between the spaces defined by the adjacent wire-like pieces are not limited in their motion by the wire-like pieces. They follow a complex up-and-down surface of the muscles. Thus, they can hold the muscle surface in close contact with it. The aforementioned predetermined distance may be, for example, from 5 mm to 1 cm. The set of wire-like pieces may be equally spaced from each other or not.

For the wire-like piece having some bends, the tight fitting band can follow and hold a complex up-and-down surface of the muscles in closer contact with it than the flat piece can do. In addition, such a wire-like piece can be attached to the tight fitting band more easily than the set of wire-like pieces, cutting out some needs in a manufacturing process.

The wire-like piece may be made of any one of suitable materials that can limit the direction towards which said tube is allowed to inflate as said tube is filled with air, to against the muscles as determined with said tight fitting band being rest on the muscles.

The wire-like piece may be made of a metal or a resin material. Alternatively, it may be made of an elastic material.

The present invention also provides a muscle development device as follows.

More specifically, the present invention provides a muscle development device comprising a hollow tight fitting band having a tube provided therein; and fastening means for use in keeping a length of the tight fitting band in a loop having a desired size, the muscle development device being used to develop muscles of a limb while restricting the blood flow therethrough by means of applying, with said tight fitting band being wrapped around a predetermined compressed range of said limb and said tight fitting band being fastened with said fastening means to have a desired size, a predetermined pressure to said limb around which said tight fitting band is wrapped, the pressure being produced by introducing air to said tube, said tube having a higher stretching rate on the side facing to the muscles than on the side opposite to said muscles, as determined with said tight fitting band being rest on the muscles, said tube being designed to inflate more in a direction against the muscles than in a direction away from the muscles as said tube is filled with air with said tight fitting band being rest on the muscles.

The aforementioned muscle development device has a wire-like piece or pieces as the means to limit the direction towards which the tube is allowed to inflate, to inward, whereas this muscle development device provides a configuration of the tube that allows the tube to inflate inward.

This muscle development device eliminates the necessity of the aforementioned flat piece for use in limiting the direction towards which the tube is allowed to inflate because the configuration of the tube itself allows the tube to inflate inward. Thus, a sufficient pressure can be applied to every part of the limb intended to be compressed, even under the influence of muscle movements.

The tube in this muscle development device may be any one of suitable tubes as long as it has a higher stretching rate on the side facing to the muscles than on the side opposite to said muscles, as determined with said tight fitting band being rest on the muscles, and it inflates more in a direction against the muscles than in a direction away from the muscles as said tube is filled with air with said tight fitting band being rest on the muscles.

For example, the tube may be made of an elastic body. The elastic body is thinner on the side facing to the muscles than on the side opposite to said muscles, as determined with said tight fitting band being rest on the muscles. The tube inflates more on the thinner side, i.e., in a direction against the muscles as the tube is filled with air. The pressure produced by the inflation of the tube can be applied to the muscles, placing an appropriate compression force onto the muscles. The elastic body may be, for example, a rubber.

The tube may be a bonded combination of elastic bodies having different stretching rates from each other in which the elastic body on the side facing to the muscles has a higher stretching rate than the elastic body on the side opposite to said muscles, as determined with said tight fitting band being rest on the muscles. The tube inflates more on the side having a higher stretching rate, i.e., in a direction against the muscles as the tube is filled with air. The pressure produced by the inflation of the tube is applied to the muscles, placing an appropriate compression force onto the muscles.

The number of the elastic bodies bonded may be two or more.

For example, the tube may be a combination of two strip-shaped elastic bodies having different stretching rates from each other, bonded along the sides thereof. Alternatively, the elastic bodies bonded may have a rectangular shape having a pair of sides along the lengthwise direction of the tight fitting band is longer than the other pair of sides. The sides of two or more rectangular elastic bodies that are parallel to the lengthwise direction of the tight fitting band may be bonded to each other and the remaining pair(s) of sides thereof may also be bonded to produce an air-tight tube.

The tube may have a higher stretching rate on the side facing to the muscles than on the side opposite to the muscles, as determined with the tight fitting band being rest on the muscles by means of adhering a seam tape having a stretching rate that is lower than that of the tube material, onto a portion of the tube on the side opposite to the muscles. The tube inflates more on the side having a higher stretching rate, i.e., in a direction against the muscles as the tube is filled with air. The pressure produced by the inflation of the tube is applied to the muscles, placing an appropriate compression force onto the muscles.

The aforementioned muscle development device further comprises air limiting means that delimits the portion of said tube into which air is allowed to enter when attached to said tight fitting band at a certain position along the length thereof.

The muscle development device having such air limiting means can delimit the portion of the tube into which air is allowed to enter according to the circumference of the limb having a compressed range to apply a compression force suitable for the compressed range. Thus, this muscle development device can be used for limbs of different circumferences. An appropriate compression force can be applied to different ranges or different individuals. Likewise, an appropriate compression force can be applied in a consistent way even when the circumference of the limb has changed because of the effect of the muscle development device. A single tight fitting band may be used widely and continuously.

### BRIEF DESCRIPTION OF THE DRAWINGS

FIG. 1 is a perspective view of a muscle development device according to a first embodiment of the present invention, a pump, and an air pressure gauge, which are connected to each other through connecting means;

FIG. 2 is a cross-sectional view of a tight fitting band of the muscle development device according to the first embodiment of the present invention;

FIG. 3A is a horizontal cross-sectional view of an outer fabric of the tight fitting band of the muscle development device according to the first embodiment of the present invention;

FIG. 3B is a vertical cross-sectional view of an outer fabric of the tight fitting band of the muscle development device according to the first embodiment of the present invention;

FIG. 4A is a view showing a clip in the muscle development device according to the first embodiment of the present invention;

FIG. 4B is a view illustrating how the clip is attached to the muscle development device according to the first embodiment of the present invention;

FIG. 5 is a perspective view illustrating the use of the muscle development device according to the first embodiment of the present invention;

FIG. 6 is a cross-sectional view of a predetermined compressed range in a case where the muscle development device according to the first embodiment of the present invention is wrapped around the compressed range;

FIG. 7 is a cross-sectional view of a portion of a tight fitting band of a muscle development device according to a second embodiment of the present invention that is located opposite to the muscles;

FIG. 8 is a cross-sectional view of a tube in a muscle development device according to a third embodiment of the present invention;

FIG. 9 is a cross-sectional view of a tube in a muscle development device according to a fourth embodiment of the present invention;

FIG. 10 is a cross-sectional view of a tube in a muscle development device according to a fifth embodiment of the present invention; and

FIG. 11 is a cross-sectional view of a predetermined compressed range in a case where a muscle development device having a flat piece is wrapped around the compressed range.

### BEST MODE FOR CARRYING OUT THE INVENTION

Referring to the drawings, embodiments of the present invention are described. In the following description of the embodiments, similar components and parts are depicted by the like reference numerals, and any redundant description will be omitted.

### First Embodiment

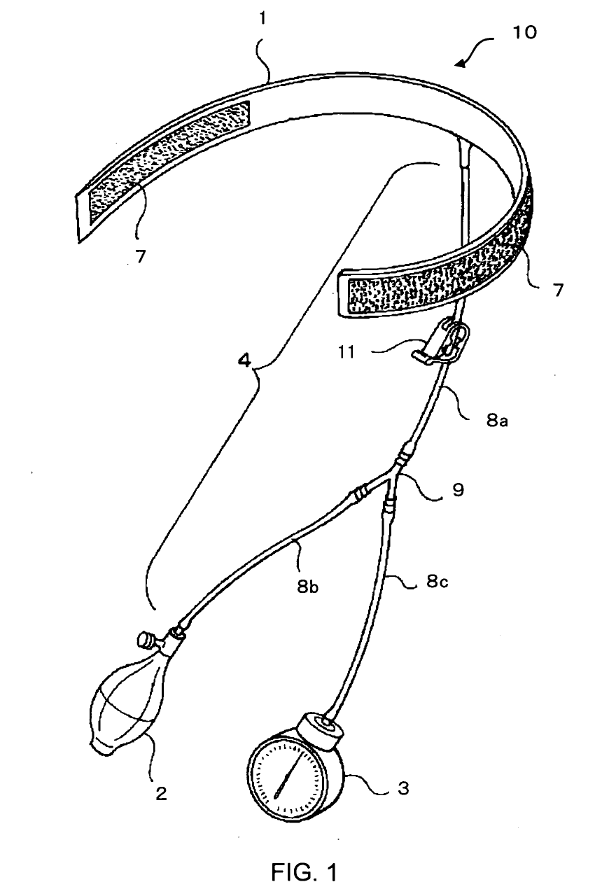

FIG. 1 is a perspective view of a muscle development device 10 according to a first embodiment of the present invention, a pump 2, and an air pressure gauge 3, which are connected to each other through connecting means 4.

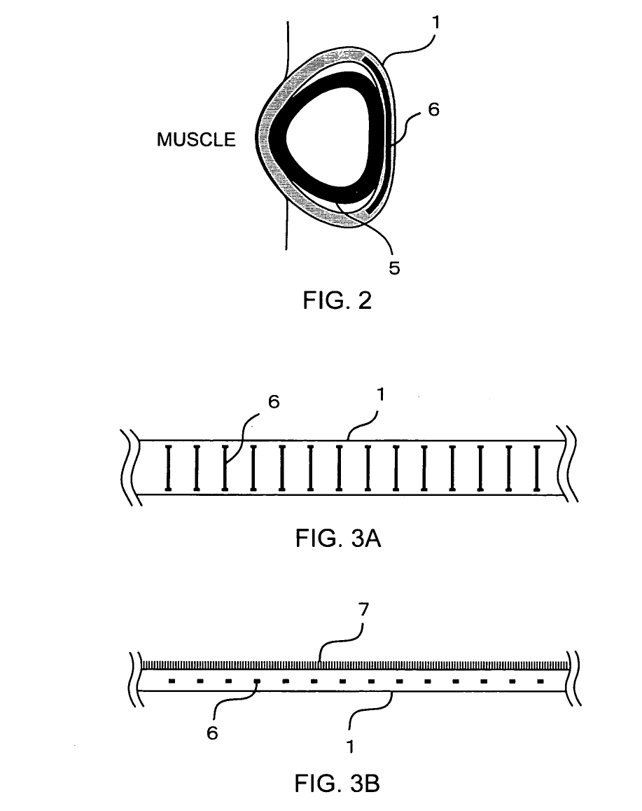

FIG. 2 is a cross-sectional view of a tight fitting band 1 in the muscle development device 10 according to this embodiment. The tight fitting band 1 is made up of two pieces of heavy fabric having a width of on the order of 5 cm. These pieces are stitched together along the lengthwise edges to form a long hollow object having a space inside it.

The tight fitting band 1 has a tube 5 therein. The tube 5 is made of a rubber that can withstand a pneumatic pressure of on the order of 200 mmHg.

The fabric used on the side facing to muscles (inner side) of the tight fitting band 1 is made of stretch threads woven together to have a net-like appearance.

The fabric used on the side opposite to muscles (outer side) is made from polyester foam. A plurality of plastic wire-like pieces 6 are provided therein generally in parallel to the widthwise direction of the tight fitting band 1 at a predetermined distance (5 mm to 1 cm) along the length of the tight fitting band 1, as shown in FIGS. 3A and 3B.

The wire-like pieces 6 in this embodiment are described as being placed generally parallel to the widthwise direction of the tight fitting band 1 but they are not limited thereto. The wire-like pieces 6 may be any similar pieces that are provided in a direction not parallel to the lengthwise direction of the tight fitting band 1 at a predetermined distance along the length of the tight fitting band 1.

The wire-like pieces 6 in this embodiment are made of a plastic material but they are not limited thereto. They may be made of any other suitable material such as a metal or a resin.

The wire-like pieces 6 in this embodiment are described as being embedded in the outer segment of the tight fitting band 1 opposite to the muscles, but they are not limited thereto. They may be provided on the inner surface of the outer segment of the tight fitting band 1 opposite to the muscles and just outside the tube 5.

For example, a plurality of wire-like pieces 6 may be placed on the inner surface of the outer segment of the tight fitting band 1 opposite to the muscles, generally in parallel to the widthwise direction of the tight fitting band 1 at a predetermined distance (5 mm to 1 cm) along the length of the tight fitting band 1. The wire-like pieces 6 may be fastened to the inner surface of the outer segment of the tight fitting band 1 opposite to the muscles, with a stretchable seam tape.

The reference numeral 7 represents a length of tape that serves as the fastening means.

The reference numeral 8 represents a connecting pipe constituting the connecting means 4. The reference numerals 8a, 8b, and 8c represent connecting pipes that are connected to the tube 5 within the tight fitting band 1, the pump 2, and the air pressure gauge 3, respectively. The connecting pipes 8a, 8b, and 8c are each a rubber-made hollow object. The reference numeral 9 represents a connection member that provides connection among the connecting pipes 8a, 8b, and 8c. It is a hollow Y-joint.

The reference numeral 11 represents a pinch clip 11 that is used to maintain the air pressure within the tube 5 by squeezing the connecting pipe 8a with it.

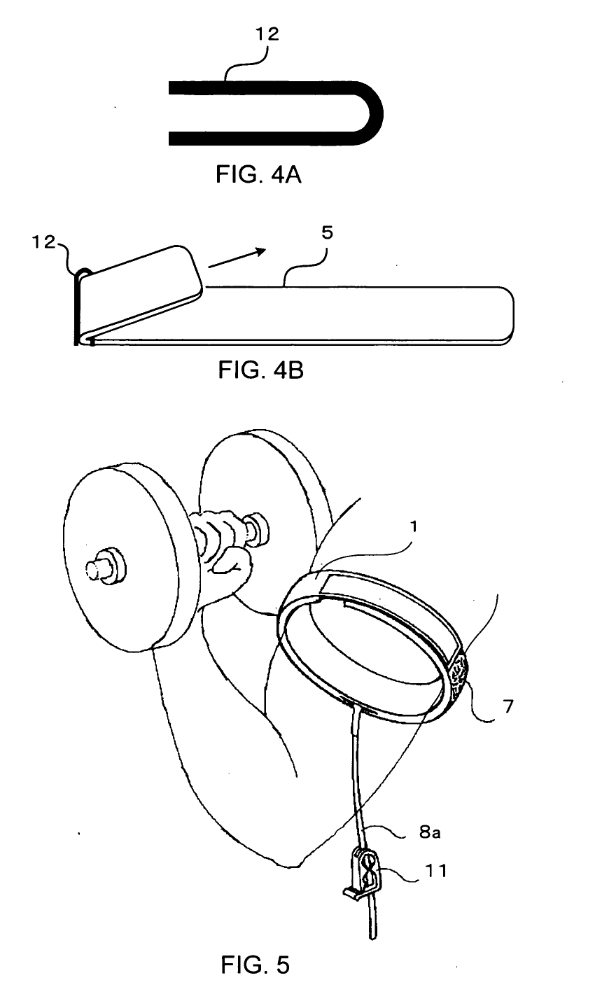

The muscle development device 10 in this embodiment also has a clip 12 as shown in FIG. 4A for delimiting the portion of the tube 5 into which air is allowed to enter when attached to the tight fitting band 1 at a certain position along the length thereof.

The clip 12 has a shape of hairpin having two parallel straight segments and another segment connecting the one end of these two straight segments with each other. The length of the straight segment of the clip 12 is slightly longer than the width of the tube 5. The distance between the straight segments is slightly shorter than the thickness of the tube 5 not being filled with the air.

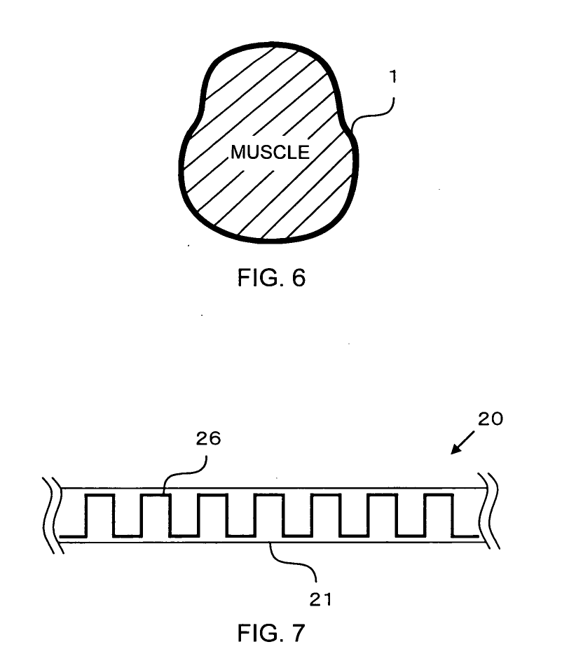

Next, how the muscle development device 10 is used is described with reference to FIG. 5.

For the muscle development with the muscle development device 10 according to the present invention, the tight fitting band 1 is put around a proximal region of the muscles that you want to develop. FIG. 5 shows an example where the biceps brachii is the target muscles for development. The tight fitting band 1 is rest on the biceps brachii at a position closer to the heart.

When the tube 5 is longer than the outer periphery of a compressed range on a limb, an extra length of the tube 5 at one end thereof may surround the compressed range and overlap with the opposite end. This can result in an improper application of the pressure. For the avoidance of such a problem, the clip 12 is attached to the tube 5 at a certain position along the length thereof to delimit the extent of the tube 5 into which the air is allowed to enter to have a generally same length as the circumference of the compressed range. Thus, a proper pressure can be applied according to the circumference of the limb having the compressed range.

To attach the clip 12 to the tube 5, the tube 5 is inserted between the straight segments of the clip 12, as shown in FIG. 4B, and the clip 12 is then fastened at a position corresponding to the extremity of the portion of the tube 5 into which the air is allowed to enter, relative to the lengthwise direction thereof.

Next, the tight fitting band 1 is fastened with the fastening means 7 to prevent the size of the loop of the tight fitting band 1 from being changed.

A gap between the arm and the tight fitting band 1, if present, upsets the relationship between the air pressure applied to the tube 5 within the tight fitting band 1 and the compression force produced by the tight fitting band 1. Thus, the tight fitting band 1 should be closely fitted to the arm without any gap.

Then, the pump 2 is used to force the air into the tube 5. The air is introduced until the pressure reaches an appropriate level while monitoring the air pressure within the tube 5 using the air pressure gauge 3.

The tube 5 being filled with the air inflates entirely, i.e., inwardly as well as outwardly. The outer half of the tube 5 is, however, pushed back by the wire-like pieces 6 that are provided outside the tube 5. Consequently, the direction towards which the tube 5 is allowed to inflate is limited to inward, as shown in FIG. 2. This brings about the application of a proper pressure to a predetermined compressed range on the limb.

The muscle development device according to this embodiment uses a plurality of wire-like pieces 6 as the means to limit the direction towards which the tube 5 is allowed to inflate, to inward, when the tube 5 is being filled with air. The adjacent wire-like pieces 6 of the plurality of wire-like pieces 6 are spaced from each other at a predetermined distance. The portions of the tube 5 that fall between the adjacent wire-like pieces 6 are not limited in their motion by the wire-like pieces 6. It can follow a complex up-and-down surface of the muscles. Therefore, as shown in FIG. 6, the tight fitting band 1 can follow and hold a complex up-and-down surface of the muscles in close contact with it in a flexible manner. When a user does exercises with the tight fitting band 1 being rest on his or her muscles, the internal pressure within the tube 5 can be kept constant and a sufficient pressure can be applied to every part even under the influence of muscle movements.

After the internal pressure within the tube 5 reaches an appropriate level, the user may either keep rest for a while or do exercises using the muscles that he or she wants to develop in order to put a stress on the muscles. It is obvious that exercises provide a much better muscle development effect, but it has been found that a muscle development effect can be obtained even when the user keeps rest without any exercises.

With some exercises of the muscles, the connecting pipe 8a is closed with the pinch clip 11 to prevent the air from escaping from the tube 5 in the tight fitting band 1 before the connecting pipe 8a is separated from the connection member 9. This is illustrated in FIG. 5.

In this embodiment, the air is described as being forced manually into the tube 5 by using the pump 2 but it is not limited thereto. The air may be introduced automatically to a desired pressure level by using a suitable device or machine such as an automatic air supply device.

### Second Embodiment

Next, a muscle development device according to a second embodiment of the present invention is described with reference to FIG. 7. A muscle development device 20 in this embodiment is basically similar to the aforementioned muscle development device 10 in the first embodiment except for the configuration of a wire-like piece 26.

FIG. 7 is a cross-sectional view of a portion of a tight fitting band 21 of the muscle development device 20 in this embodiment that is located opposite to the muscles.

The wire-like piece 26 used in the muscle development device in this embodiment is formed by bending a single wire-like piece.

More specifically, the wire-like piece 26 in this embodiment is formed by bending a single wire-like piece to put a series of angles in it and produce segments that are generally parallel to the widthwise direction of the tight fitting band 21 at a predetermined distance, as shown in FIG. 7.

The requirement for the wire-like piece 26 is that it is formed to have a segment that is not parallel to the lengthwise direction of the tight fitting band 21. For example, a wire-like piece formed into a zigzag pattern like a continuous series of “V”s may be used.

This wire-like piece 26 may be embedded in the outer segment of the tight fitting band 21 opposite to the muscles or may be provided on the inner surface of the outer segment of the tight fitting band 21 opposite to the muscles and just outside the tube 5, as in the first embodiment.

The wire-like piece 26 in this embodiment is a single wire-like piece that is bent into meander. This eliminates the necessity of placing the wire-like pieces at a certain distance along the direction generally parallel to the widthwise direction of the tight fitting band, as opposed to the case of the wire-like pieces 6 in the first embodiment. Attachment to the tight fitting band is easier as compared with the wire-like pieces 6 in the first embodiment.

With the wire-like piece 26 of the type described, it can hold a complex up-and-down surface of the muscles in closer contact with it than the flat piece can do. Therefore, this provides similar effects to those obtained with the muscle development device in the first embodiment.

### Third Embodiment

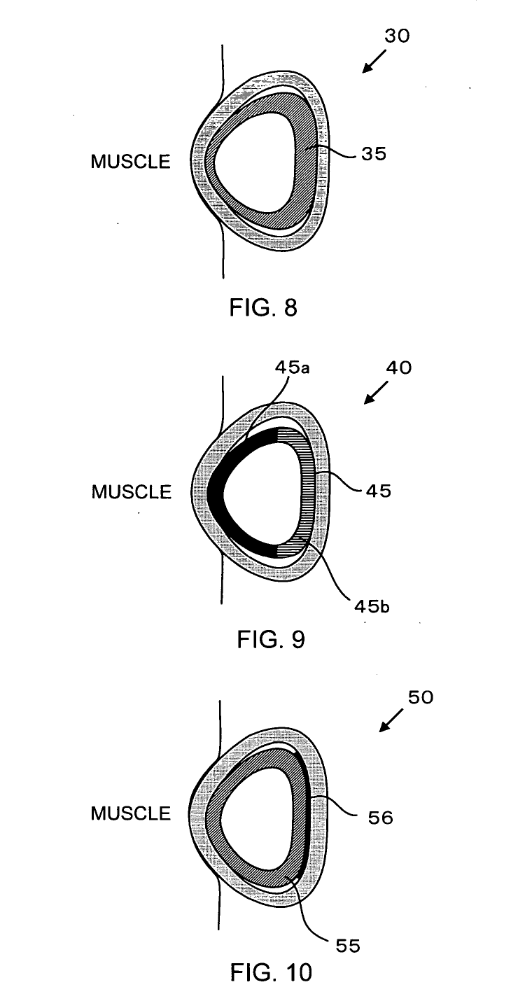

Next, a muscle development device according to a third embodiment of the present invention is described with reference to FIG. 8.

A muscle development device 30 in this embodiment is basically similar to the muscle development devices in the aforementioned embodiments. The major difference lies in the following. The muscle development devices in the aforementioned embodiments comprise a wire-like piece or pieces as the means to limit the direction towards which the tube is allowed to inflate, to inward, whereas in the muscle development device according to this embodiment the tube itself is configured in such a manner that the direction towards which the tube is allowed to inflate is directed to inward. The requirement for the tube is that the tube is designed to have a higher stretching rate on the side facing to the muscles than on the side opposite to the muscles, as determined with the tight fitting band being rest on the muscles, and it is also designed to inflate more in a direction against the muscles as the tube is filled with air with the tight fitting band being rest on the muscles.

A tube 35 in this embodiment is made of a rubber. The part of the tube 35 located on the side facing to the muscles is thinner than the part located on the side opposite to the muscles, as determined with the tight fitting band being rest on the muscles.

In the muscle development device 30 having such the tube 35, the tube 35 inflates more on the thinner side, i.e., in a direction against the muscles as the tube 35 is filled with air. The pressure produced by the inflation of the tube 35 can be applied to the muscles without using the wire-like piece(s), placing an appropriate compression force onto the muscles. This eliminates any means to limit the direction towards which the tube 35 is allowed to inflate, to inward. Any drawbacks associated with the flat piece can also be eliminated.

In other words, it can follow and hold a complex up-and-down surface of the muscles in close contact with it in a flexible manner. When a user does exercises with the tight fitting band 1 being rest on his or her muscles, the internal pressure within the tube 35 can be kept constant and a sufficient pressure can be applied to every part even under the influence of muscle movements. This eliminates any means to limit the direction towards which the tube 35 is allowed to inflate, to inward, providing a tight fitting band having a simple configuration.

### Fourth Embodiment

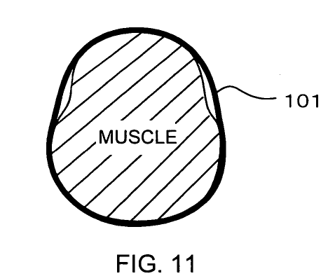

Next, a muscle development device according to a fourth embodiment of the present invention is described with reference to FIG. 9.

A muscle development device 40 in this embodiment is basically similar to the muscle development device 30 in the third embodiment except for a unique feature of the tube.

A tube 45 in this embodiment is a combination of two strip-shaped elastic bodies 45a and 45b having different stretching rates from each other, bonded along the sides thereof. The elastic body 45a located on the side facing to the muscles has a higher stretching rate than the elastic body 45b located on the side opposite to the muscles, as determined with the tight fitting band being rest on the muscles.

The tube 45 configured as described above has a higher stretching rate on the side facing to the muscles than on the side opposite to the muscles, as determined with the tight fitting band being rest on the muscles. The tube 45 inflates more in a direction against the muscles as the tube 45 is filled with air with the tight fitting band being rest on the muscles.

### Fifth Embodiment

Next, a muscle development device according to a fifth embodiment of the present invention is described with reference to FIG. 10.

A muscle development device 50 in this embodiment is basically similar to the muscle development devices in the third and fourth embodiments except for the structure of the tube.

The muscle development device in this embodiment also provides a unique feature of the tube as in the muscle development devices in these embodiments.

A tube 55 in this embodiment has a uniform stretching rate over the entire surface thereof. A seam tape 56, which is stretchable but of which stretching rate is lower than that of the tube 55, is adhered to the tube 55 on the side opposite to the muscles. It is designed to have a higher stretching rate on the side facing to the muscles than on the side opposite to the muscles, as determined with the tight fitting band being rest on the muscles.

The tube 55 configured as described above has a higher stretching rate on the side facing to the muscles than on the side opposite to the muscles, as determined with the tight fitting band being rest on the muscles. The tube 55 inflates more in a direction against the muscles as the tube 55 is filled with air with the tight fitting band being rest on the muscles.

1. A muscle development device comprising:
a hollow tight fitting band having a tube and a wire-like piece provided therein; and
fastening means for use in keeping a length of the tight fitting band in a loop having a desired size,
the muscle development device being used to develop muscles of a limb while restricting the blood flow therethrough by means of applying, with said tight fitting band being wrapped around a predetermined compressed range of said limb and said tight fitting band being fastened with said fastening means to have a desired size, a predetermined pressure to said limb around which said tight fitting band is wrapped, the pressure being produced by introducing air to said tube,
said wire-like piece being designed to limit the direction towards which said tube is allowed to inflate as said tube is filled with air, to against the muscles as determined with said tight fitting band being rest on the muscles.

2. The muscle development device as claimed in claim 1, wherein said wire-like piece has a segment that is not parallel to the lengthwise direction of said tight fitting band.

3. The muscle development device as claimed in claim 2, wherein said wire-like piece is a set of wire-like pieces that are placed in a direction not parallel to the lengthwise direction of said tight fitting band at a predetermined distance along the length of said tight fitting band.

4. The muscle development device as claimed in claim 2, wherein said wire-like piece is made of a single wire-like piece having a bend or a series of bends.

5. A muscle development device comprising:
a hollow tight fitting band having a tube provided therein; and
fastening means for use in keeping a length of the tight fitting band in a loop having a desired size,
the muscle development device being used to develop muscles of a limb while restricting the blood flow therethrough by means of applying, with said tight fitting band being wrapped around a predetermined compressed range of said limb and said tight fitting band being fastened with said fastening means to have a desired size, a predetermined pressure to said limb around which said tight fitting band is wrapped, the pressure being produced by introducing air to said tube,
said tube having a higher stretching rate on the side facing to the muscles than on the side opposite to said muscles, as determined with said tight fitting band being rest on the muscles,
said tube being designed to inflate more in a direction against the muscles than in a direction away from the muscles as said tube is filled with air with said tight fitting band being rest on the muscles.

6. The muscle development device as claimed in claim 5, wherein said tube is made of an elastic body, the elastic body being thinner on the side facing to the muscles than on the side opposite to said muscles, as determined with said tight fitting band being rest on the muscles.

7. The muscle development device as claimed in claim 5, wherein said tube is a bonded combination of elastic bodies having different stretching rates from each other, the elastic body on the side facing to the muscles having a higher stretching rate than the elastic body on the side opposite to said muscles, as determined with said tight fitting band being rest on the muscles.

8. The muscle development device as claimed in claim 1, wherein further comprises air limiting means that delimits the portion of said tube into which air is allowed to enter when attached to said tight fitting band at a certain position along the length thereof.
